(19) United States
(12) Patent Application Publication
(10) Pub. No.: US 2026/0188511 A1
(43) Pub. Date: Jul. 2, 2026

(54) RISK PREDICTION DEVICE, RISK PREDICTION METHOD, AND RECORDING MEDIUM

(71) Applicant: NEC Corporation, Tokyo (JP)

(72) Inventors: Mana HASHIMOTO, Tokyo (JP); Yuki KOSAKA, Tokyo (JP); Kosuke NISHIHARA, Tokyo (JP)

(73) Assignee: NEC Corporation, Tokyo (JP)

(21) Appl. No.: 19/419,409

(22) Filed: Dec. 15, 2025

(30) Foreign Application Priority Data
Dec. 26, 2024 (JP) 2024-230368

(51) Int. Cl. G16H 50/30; A61B 5/00; G16H 20/00; G16H 40/67; G16H 50/20

(52) U.S. Cl. CPC G16H 50/30; A61B 5/4815; A61B 5/742; G16H 20/00; G16H 40/67; G16H 50/20

(57) ABSTRACT

In order to feed back the risk of degradation of a quality of sleep to a subject, in a risk prediction device, a processor acquires a life log from the subject terminal, the life log including quality-of-sleep information and lifestyle information. The processor creates a life map representing a tendency of a lifestyle in a coordinate space having lifestyle-related parameters, and estimates a latest state by calculating the lifestyle-related parameters and mapping the latest state in the life map. The processor predicts a risk of degradation of quality, based on a positional relationship between a position of the latest state and a region in the life map, and provides feedback if the risk is equal to or more than a threshold. the subject terminal displays an alert and a recommended action to reduce the risk. Accordingly, it is possible to supports users in making decisions concerning their sleep.

### INCORPORATION BY REFERENCE

This application is based upon and claims the benefit of priority from Japanese Patent Application 2024-230368, filed on Dec. 26, 2024, the disclosure of which is incorporated herein in its entirety by reference.

### TECHNICAL FIELD

The present disclosure relates to a technique for predicting a risk of degradation of quality of sleep.

### BACKGROUND ART

Sleep is a rest activity essential for promoting and maintaining health in all ages of children, adults, and elderly people. However, the average sleep time of Japanese is the shortest among 33 countries in the world mainly including developed countries, and it can be said that ensuring of high-quality sleep is an important health issue for the people. In addition, “ensuring of appropriate sleep time” and “improvement of sleep rest feeling” are important issues that all the people should address, and are considered to be meaningful in extending the healthy life expectancy of our country. Patent Document 1 describes a terminal device that displays advice for improving the quality of sleep, based on subjective evaluation of the user regarding biological information and sleep of the user.

Patent Document 1: Japanese Patent Application Laid-Open under No. 2024-014217

### SUMMARY

It is widely known to measure sleep time and quality of sleep by a terminal device or an application. However, it has been difficult to notify a user of a risk of degradation of quality of sleep by the terminal device or the application.

One of the objects of the present disclosure is to feed back the risk of degradation of the quality of sleep to the user.

According to an example aspect of the present invention, there is provided a risk prediction device including:
at least one interface configured to communicate with a subject terminal used by a subject;
at least one memory configured to store instructions; and
at least one processor configured to execute the instructions to:
acquire, via the at least one interface, a life log for the subject from the subject terminal, the life log including at least quality-of-sleep information relating to sleep of the subject and lifestyle information for a plurality of days;
create, based on the life log, a life map representing a tendency of a lifestyle of the subject in a coordinate space having, as axes, a plurality of lifestyle-related parameters calculated from the lifestyle information;
estimate a latest state of the subject by calculating, from a latest life log of the subject for a predetermined period, values of the plurality of lifestyle-related parameters and mapping the latest state to a position in the coordinate space of the life map;
predict a risk of degradation of quality of sleep of the subject, based on a positional relationship between the position of the latest state and a region in the life map that is associated with a predetermined condition on the quality-of-sleep information included in the life log; and
control the subject terminal, via the at least one interface, to provide feedback to the subject in a case where the risk is equal to or more than a threshold, by causing the subject terminal to display at least one of an alert indicating a high possibility of low quality of sleep will be and a recommended action for changing at least one of the plurality of lifestyle-related parameters so that the risk is reduced.

According to another example aspect of the present invention, there is provided a risk prediction method executed by a risk prediction device including at least one interface configured to communicate with a subject terminal used by a subject, at least one memory configured to store instructions, and at least one processor configured to execute the instructions, the risk prediction method comprising:
acquiring, via the at least one interface, a life log for the subject from the subject terminal, the life log including at least quality-of-sleep information relating to sleep of the subject and lifestyle information for a plurality of days;
creating, based on the life log, a life map representing a tendency of a lifestyle of the subject in a coordinate space having, as axes, a plurality of lifestyle-related parameters calculated from the lifestyle information;
estimating a latest state of the subject by calculating, from a latest life log of the subject for a predetermined period, values of the plurality of lifestyle-related parameters and mapping the latest state to a position in the coordinate space of the life map;
predicting a risk of degradation of quality of sleep of the subject, based on a positional relationship between the position of the latest state and a region in the life map that is associated with a predetermined condition on the quality-of-sleep information included in the life log; and
providing feedback to the subject in a case where the risk is equal to or more than a threshold, by controlling the subject terminal via the at least one interface to display at least one of an alert indicating a high probability that the quality of sleep will be low and a recommended action for changing at least one of the plurality of lifestyle-related parameters so that the risk is reduced.

According to further example aspect of the present invention, there is provided a non-transitory computer-readable recording medium storing a program causing a computer of a risk prediction device including at least one interface configured to communicate with a subject terminal used by a subject, at least one memory configured to store instructions, and at least one processor configured to execute the instructions to execute processing of:
acquiring, via the at least one interface, a life log for the subject from the subject terminal, the life log including at least quality-of-sleep information relating to sleep of the subject and lifestyle information for a plurality of days;
creating, based on the life log, a life map representing a tendency of a lifestyle of the subject in a coordinate space having, as axes, a plurality of lifestyle-related parameters calculated from the lifestyle information;
estimating a latest state of the subject by calculating, from a latest life log of the subject for a predetermined period, values of the plurality of lifestyle-related parameters and mapping the latest state to a position in the coordinate space of the life map;
predicting a risk of degradation of quality of sleep of the subject, based on a positional relationship between the position of the latest state and a region in the life map that is associated with a predetermined condition on the quality-of-sleep information included in the life log; and
providing feedback to the subject in a case where the risk is equal to or more than a threshold, by controlling the subject terminal via the at least one interface to display at least one of an alert indicating a high probability that the quality of sleep will be low and a recommended action for changing at least one of the plurality of lifestyle-related parameters so that the risk is reduced.

### EFFECT

According to the present disclosure, it is possible to feed back the risk of degradation of the quality of sleep to the user.

### BRIEF DESCRIPTION OF THE DRAWINGS

FIG. 1 illustrates an example of a schematic configuration of a risk prediction system according to the present disclosure;

FIGS. 2A and 2B are block diagrams illustrating an example of hardware configurations of a server and a subject terminal;

FIGS. 3A and 3B illustrate an example of data stored in a personal information DB and a life log DB;

FIG. 4 illustrates an example of an input screen for quality of sleep;

FIG. 5 is a block diagram illustrating an example of a functional configuration of the server;

FIG. 6 illustrates an example of a life map with an average sleep time and an average number of steps as axes;

FIGS. 7A and 7B illustrate examples in which a life map is converted into another graph;

FIG. 8 illustrates an example in which a part of the life map is converted into another graph and enlarged;

FIG. 9 illustrates an example of a diagram for explaining deviation detection;

FIG. 10 is a flowchart illustrating an example of processing by a feedback unit;

FIG. 11 illustrates an example of a visualization map with the average sleep time and the average number of steps as axes;

FIG. 12 is a diagram for explaining an action proposal based on the visualization map;

FIG. 13 is a flowchart illustrating an example of processing by an action proposal unit;

FIGS. 14A and 14B are diagrams for explaining a plurality of routes regarding an action for reaching a target;

FIG. 15 is a flowchart illustrating an example of feedback processing;

FIGS. 16A to 16C illustrate an example of a life map in September and converted graphs;

FIG. 17 is a block diagram illustrating an example of a functional configuration of a risk prediction device; and

FIG. 18 is a flowchart illustrating an example of processing by the risk prediction device.

### EXAMPLE EMBODIMENTS

Hereinafter, example embodiments of the present disclosure will be described with reference to the drawings.

### First Example Embodiment

### Overall Configuration

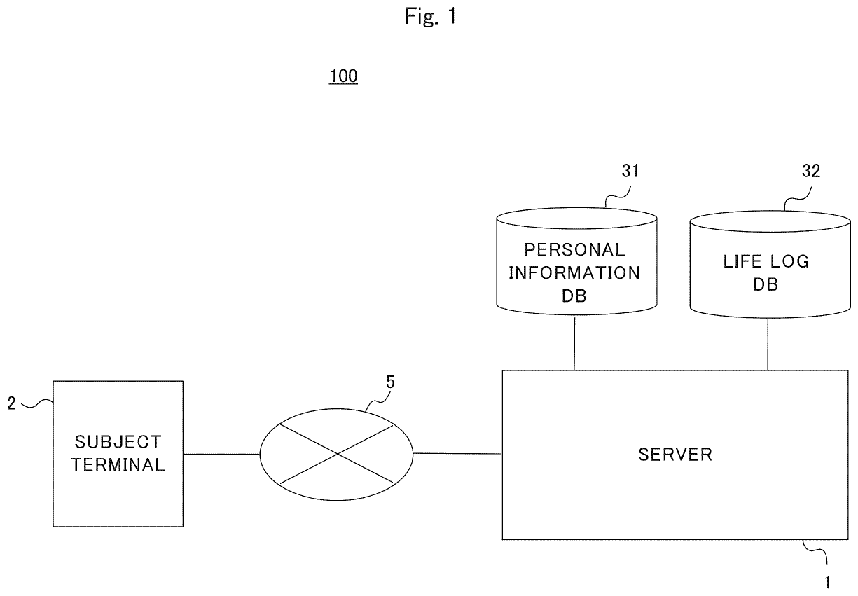

FIG. 1 illustrates an example of a schematic configuration of a risk prediction system 100 to which a risk prediction device of the present disclosure is applied. The risk prediction system 100 is a system for estimating a tendency of a lifestyle, based on a life log of a subject, to predict a risk of degradation of quality of sleep (Hereinafter, it is also simply referred to as “risk”.), and feeding back an alert or an action for improving the quality of sleep (Hereinafter, it is also referred to as a “recommended action”.) to the subject in a case where the risk is high.

In the risk prediction system 100 in FIG. 1, a server 1 and a subject terminal 2 are communicably connected to each other via a network 5 such as the Internet. The risk prediction system 100 provides, for a plurality of users, a service of predicting a risk to output an alert and propose a recommended action in a case where the risk is high. With this service, the users can improve the quality of sleep and promote health. The subject is one of the users who enjoy the service provided by the risk prediction system 100. In FIG. 1, for convenience, the server 1 is connected to one subject terminal 2, but is actually connected to a plurality of terminal devices respectively used by the plurality of users.

The subject terminal 2 is a smartphone, a smart watch, or the like used by the subject, acquires a life log of the subject and transmits the life log to the server 1, or receives an alert or a recommended action from the server 1. The subject terminal 2 is an example of a terminal used by the subject of the present disclosure.

The server 1 is an information processing device that processes, stores, and transmits/receives various data, and is connected to a personal information database (Hereinafter, a “database” is referred to as a “DB”.) 31 and a life log DB 32. The server 1 receives a life log of the subject from the subject terminal 2, predicts a risk, based on the life log, and transmits an alert or a recommended action to the subject terminal 2 in a case where the risk is high. The server 1 may be a virtual server in a cloud environment. The server 1 is an example of the risk prediction device of the present disclosure.

### Hardware Configuration

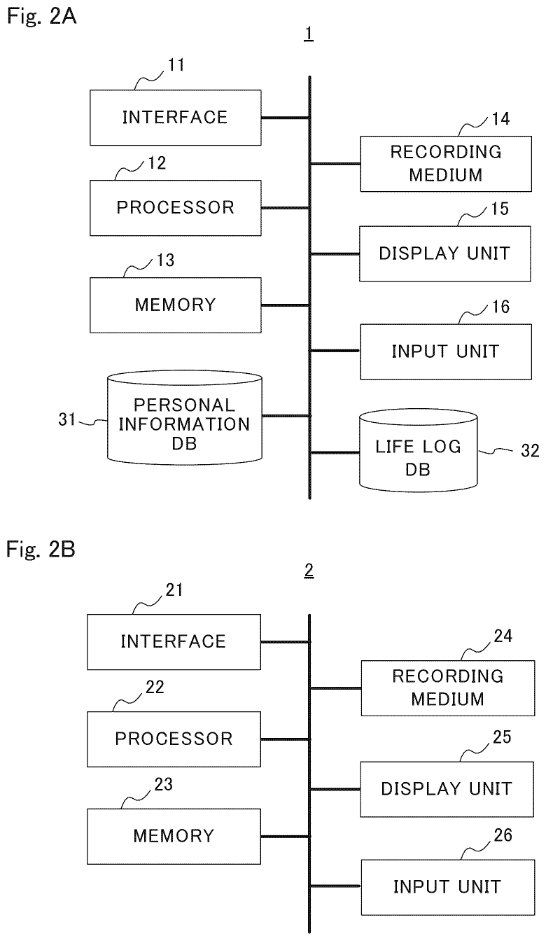

FIG. 2A is a block diagram illustrating an example of a hardware configuration of the server 1. As illustrated in FIG. 2A, the server 1 includes an interface 11, a processor 12, a memory 13, a recording medium 14, a display unit 15, and an input unit 16. These constituent elements, the personal information DB 31, and the life log DB 32 are connected to each other via a bus.

The interface 11 exchanges data with the subject terminal 2. The interface 11 is used in a case of receiving a life log from the subject terminal 2, or transmitting an alert or a recommended action to the subject terminal 2 in a case where the risk is high.

The processor 12 is a computer such as a Central Processing Unit (CPU), and controls the entire server 1 by executing a program prepared in advance. As the processor 12, it is possible to use a CPU, a Graphics Processing Unit (GPU), a Digital Signal Processor (DSP), a Micro Processing Unit (MPU), a Floating Point Unit (FPU), a Physics Processing Unit (PPU), a Tensor Processing Unit (TPU), a quantum processor, a microcontroller, a combination of these, or the like.

The memory 13 includes a Read Only Memory (ROM), a Random Access Memory (RAM), and the like. The memory 13 stores a program executed by the processor 12. The memory 13 is also used as a work memory during execution of various types of processing by the processor 12.

The recording medium 14 is a non-volatile and non-transitory recording medium such as a disk-shaped recording medium or a semiconductor memory, and is attachable to and detachable from the server 1. The recording medium 14 records various programs executed by the processor 12. In a case where the server 1 executes feedback processing, the program recorded in the recording medium 14 is loaded into the memory 13 and executed by the processor 12.

The display unit 15 displays a predetermined image by, for instance, a Liquid Crystal Display (LCD). The input unit 16 is a keyboard, a mouse, a touch panel, or the like, and is used by an operator who manages the server 1.

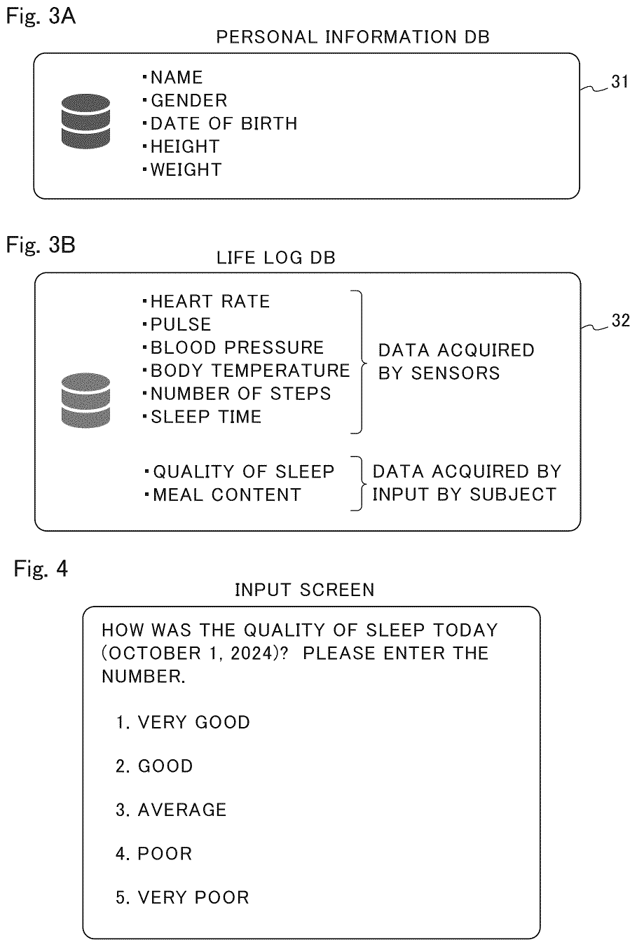

FIG. 3A is an example schematically illustrating data stored in the personal information DB 31. The personal information DB 31 stores, as personal information, for instance, the name, gender, date of birth, height, and weight of each user registered at the time of user registration of the service, in association with an identification ID for identifying the user.

FIG. 3B is an example schematically illustrating data stored in the life log DB 32. For instance, the life log DB 32 associates a heart rate, pulse, blood pressure, body temperature, number of steps, sleep time, quality of sleep, and meal content of each user with the identification ID together with time information, and stores them as a life log. As described above, the life log includes information regarding parameters representing the tendency of the lifestyle such as the number of steps and the sleep time of each user. The parameters included in the life log are not limited to the example in FIG. 3B, and can be arbitrarily set if the parameters represent the tendency of the lifestyle of the user.

The data of the heart rate, pulse, blood pressure, body temperature, number of steps, and sleep time are measured and acquired by a predetermined application installed in the subject terminal 2 using sensors. The time information is a date and time in a case where each of the heart rate, pulse, blood pressure, body temperature, number of steps, and sleep time is measured. The quality of sleep and the meal content are acquired by the subject inputting them on an input screen displayed by a predetermined application installed in the subject terminal 2. The time information regarding the quality of sleep is a date and time in a case where sleep is taken, and the time information regarding the meal content is a date and time in a case where a meal is taken. The subject terminal 2 transmits the acquired life log to the server 1 at any time, and the server 1 stores the life log in the life log DB 32 if the life log is acquired from the subject terminal 2.

FIG. 4 illustrates an example of an input screen for quality of sleep. As illustrated in FIG. 4, the input screen includes a message “How was the quality of sleep today (October 1, 2024)? Please enter the number.” and “1 very good”, “2 good”, “3 average”, “4 poor”, and “5 very poor” for evaluating the quality of sleep. For instance, the subject inputs subjective evaluation of the quality of sleep in numbers by a predetermined operation on the input screen displayed on the subject terminal 2 at a timing of getting up. As a result, the subject terminal 2 can acquire the quality of sleep as a life log.

The input screen in FIG. 4 is an example, and as long as subjective evaluation of the quality of sleep by the user can be acquired, a configuration of the input screen and an acquisition method are not limited thereto, and can be arbitrarily set.

FIG. 2B is a block diagram illustrating an example of a hardware configuration of the subject terminal 2. As illustrated in FIG. 2B, the subject terminal 2 includes an interface 21, a processor 22, a memory 23, a recording medium 24, a display unit 25, and an input unit 26.

The interface 21 exchanges data with the server 1 via the network 5. The interface 21 is used in a case of transmitting a life log of the subject to the server 1, or receiving an alert or a recommended action from the server 1.

The processor 22 is a computer such as a CPU, and controls the entire subject terminal 2 by executing a program prepared in advance. As the processor 22, it is possible to use a CPU, a GPU, a DSP, an MPU, an FPU, a PPU, a TPU, a quantum processor, a microcontroller, a combination of these, or the like. For instance, in a case where the subject terminal 2 is a smart watch, the smart watch is worn by the subject and a predetermined program is executed, whereby it is possible to acquire the heart rate, pulse, blood pressure, body temperature, and the like of the subject.

The memory 23 includes a ROM, a RAM, and the like. The memory 23 stores a program executed by the processor 22. The memory 23 is also used as a work memory during execution of various types of processing by the processor 22.

The recording medium 24 is a non-volatile non-transitory recording medium such as a disk-shaped recording medium or a semiconductor memory, and is attachable to and detachable from the subject terminal 2. The recording medium 24 records various programs executed by the processor 22. The display unit 25 displays a predetermined image by, for instance, an LCD. The input unit 26 is a touch panel or the like, and is used in a case where the subject performs a predetermined operation.

### Functional Configuration

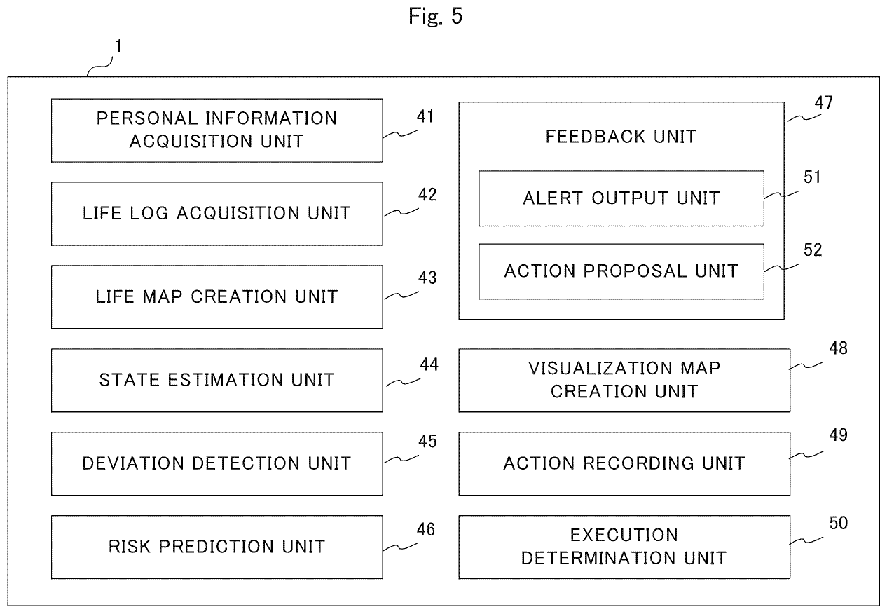

FIG. 5 is a block diagram illustrating an example of a functional configuration of the server 1. The server 1 functionally includes a personal information acquisition unit 41, a life log acquisition unit 42, a life map creation unit 43, a state estimation unit 44, a deviation detection unit 45, a risk prediction unit 46, a feedback unit 47, a visualization map creation unit 48, an action recording unit 49, and an execution determination unit 50.

The personal information acquisition unit 41, the life log acquisition unit 42, the life map creation unit 43, the state estimation unit 44, the deviation detection unit 45, the risk prediction unit 46, the feedback unit 47, the visualization map creation unit 48, the action recording unit 49, and the execution determination unit 50 are implemented by the processor 12 executing a program.

The personal information acquisition unit 41 acquires, for instance, the gender and date of birth of the subject from the personal information DB 31, based on the identification ID of the subject.

The life log acquisition unit 42 acquires, for instance, the quality of sleep, sleep time, and number of steps of the subject from the life log DB 32, based on the identification ID of the subject together with respective pieces of the time information. The life log acquisition unit 42 acquires a life log of the user necessary for creating the life map from the life log DB 32.

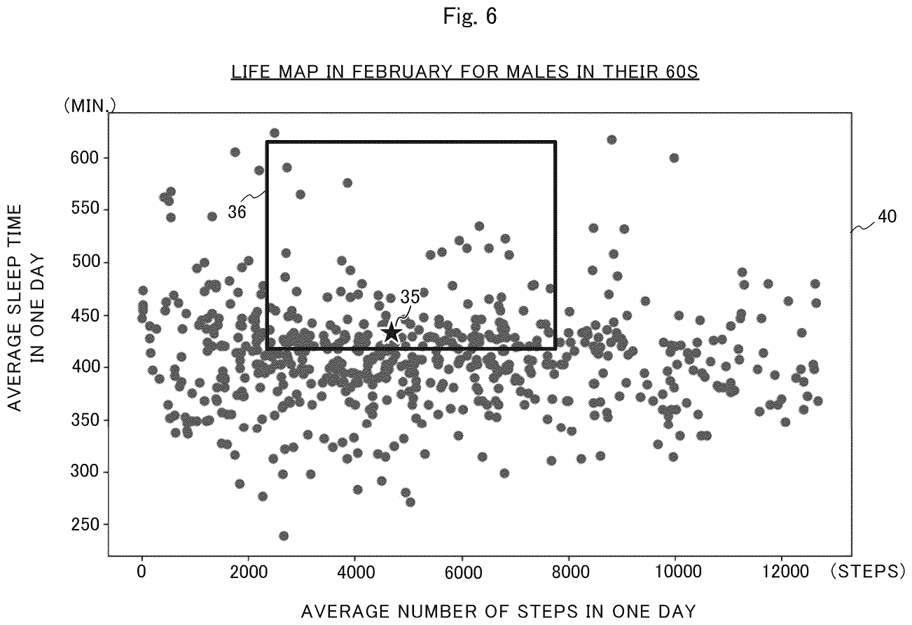

The life map creation unit 43 creates a life map representing the tendency of the lifestyle, based on the life log. Specifically, the life map creation unit 43 creates a life map representing one user as one point with the parameters included in the life log as axes. A certain period (for instance, a certain week) of one user may be represented by one point. FIG. 6 illustrates an example of a life map with an average number of steps and an average sleep time as axes. The life map 40 illustrated in FIG. 6 is a “life map in February for males in their 60s”, and the horizontal axis (x axis) represents the average number of steps and the vertical axis (y axis) represents the average sleep time, based on statistical information on life logs in February for a plurality of male users in their 60s of the same age and the same sex as the subject. The life map 40 is a density map in which one male in his 60s is represented by one point, based on the average number of steps and the average sleep time. With the life map, it is possible to estimate the tendency of the lifestyle of users of the same age and the same sex as the subject. A state of the subject himself/herself with respect to an average state of other users can be visualized with the life map.

The life map is not limited to being created based on life logs of a plurality of users of the same age and the same sex as the subject, and may be created based on life logs of the subject himself/herself in the past. In this case, for instance, the life map creation unit 43 creates, based on life logs in “February 1 to February 28” of the subject for past several years, a life map in which the horizontal axis (x axis) represents the number of steps and the vertical axis (y axis) represents the sleep time, and the subject of each date is represented by one point, based on the number of steps and the sleep time of each date. According to this, with the life map, it is possible to estimate the tendency of the lifestyle of the subject himself/herself.

Only life logs in which subjective evaluation of the quality of sleep is good are extracted, and the life map may be created based on the extracted life logs. In this case, for instance, the life map creation unit 43 extracts, from life logs in February for the users of the same age and the same sex as the subject, only life logs of users for which the subjective evaluation is input as “1 very good” or “2 good”, and creates a life map based on statistical information on the extracted life logs. According to this, with the life map, it is possible to estimate the tendency of the lifestyle of users who are of the same age and the same sex as the subject and feel that the quality of sleep is high.

For instance, the life map creation unit 43 extracts, from the life logs in “February 1 to February 28” of the subject for the past several years, only life logs of the subject on dates in a case where the subjective evaluation is input as “1 very good” or “2 good”, and creates a life map based on the extracted life logs. According to this, with the life map, it is possible to estimate the tendency of the lifestyle in a case where the subject feels that the quality of sleep is high.

In FIG. 6, as an example, the life map 40 is created in which the average number of steps and the average sleep time are set to the x axis and the y axis, respectively, but the present disclosure is not limited thereto, and the life map can be created with any parameters included in the life log as axes.

The state estimation unit 44 estimates the latest state of the subject, based on the latest life log of the subject. Specifically, the state estimation unit 44 estimates the latest state of the subject from a position on the life map, based on the parameters included in the latest life log of the subject. For instance, in a case where the latest sleep time and number of steps of the subject are 430 minutes and 4500 steps, respectively, the state estimation unit 44 estimates the latest state of the subject from a position of a star 35 on the life map illustrated in FIG. 6.

The deviation detection unit 45 detects that the latest state of the subject deviates from a normal state in the tendency of the lifestyle, and acquires deviation information regarding deviation. The normal state may be, for instance, a state corresponding to an average of a plurality of users of the same age and the same sex, or a state in which there are many users of the same age and the same sex. In a case where the life map is created based on the life logs of the subject himself/herself in the past, the normal state may be, for instance, a state corresponding to an average of states of the subject on respective dates or a state in which there are many states of the subject on respective dates.

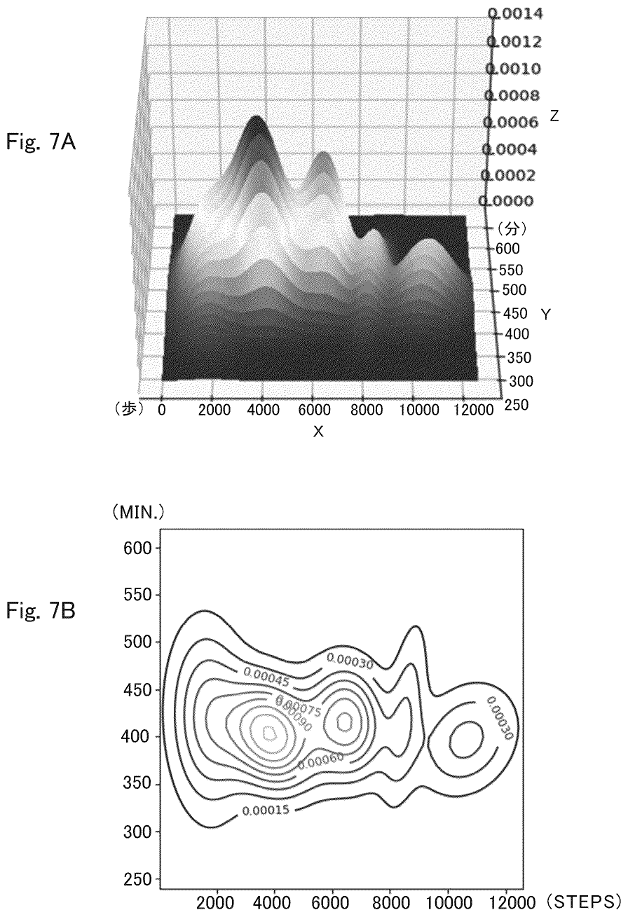

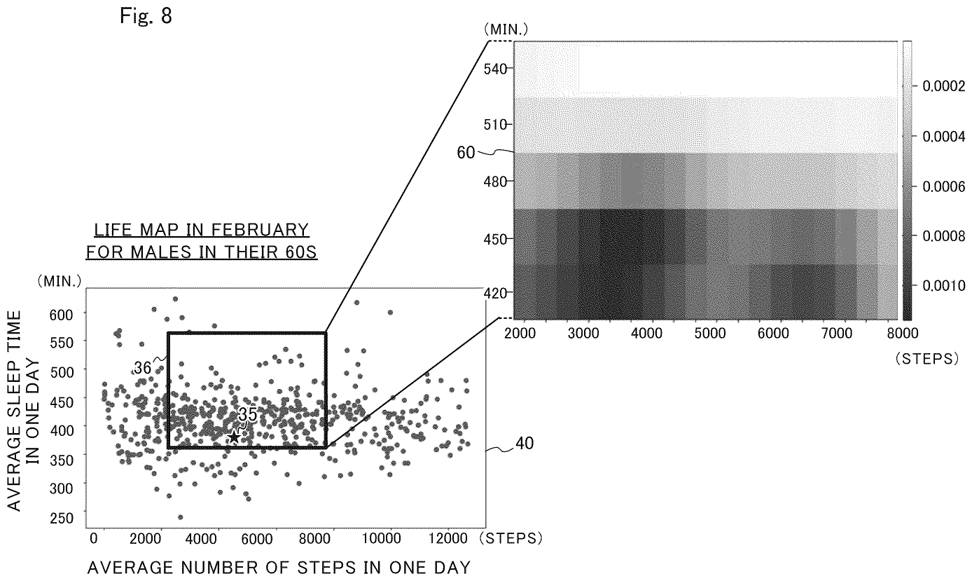

FIG. 7A illustrates an example in which the life map 40 is converted into a three-dimensional graph with density of points indicating users as the Z axis. FIG. 7B illustrates an example in which the life map 40 is converted into a graph representing the density of points indicating the users with contour lines. In the graphs illustrated in FIGS. 7A and 7B, it can be said that it is a state in which there are more users having the same average sleep time and average number of steps in a position where the peak is higher. FIG. 8 illustrates an example in which a portion surrounded by a thick line 36 of the life map 40 is converted into a graph representing the density of points indicating the users by a concentration. In a graph 60 illustrated in FIG. 8, it is a state in which there are more users having the same average sleep time and average number of steps in a position where the concentration is higher.

FIG. 7A, FIG. 7B, and FIG. 8 are all examples in which the life map 40 is converted into another graph, but for convenience of description, deviation detection will be described by using a graph representing the density of points indicating the users by concentration as illustrated in FIG. 8. It can be said that FIG. 7A, FIG. 7B, and FIG. 8 are also life maps representing the tendency of the lifestyle of the user.

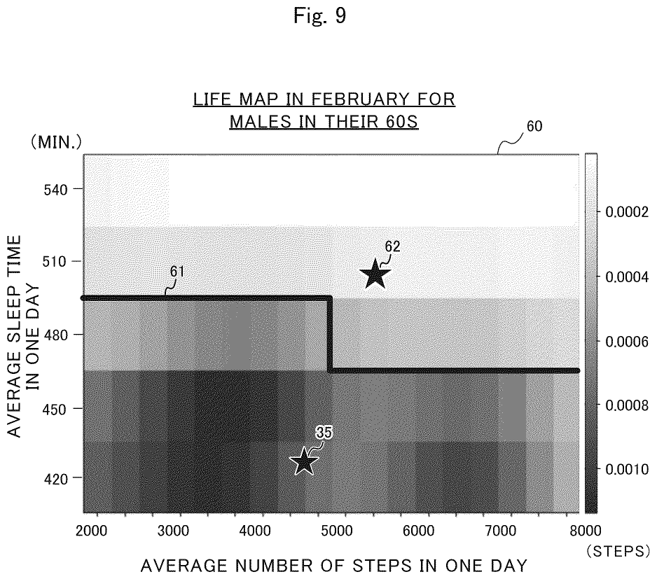

FIG. 9 illustrates an example of a diagram for explaining deviation detection. As illustrated in FIG. 9, in the graph 60, a range below a thick line 61 has a high concentration, and is a range in which there are many users having the same average sleep time and average number of steps, that is, a normal range. On the other hand, a range above the thick line 61 has a low concentration, and is a range in which there are less users having the same average sleep time and average number of steps, that is, outside the normal range. In a case where the latest state of the subject is at the position of the star 35, since the position is in an area below the thick line 61, the deviation detection unit 45 determines that the state is within the normal range, and does not detect deviation. On the other hand, in a case where the latest state of the subject is at a position of a star 62, since the position is in an area above the thick line 61, the deviation detection unit 45 determines that the state is outside the normal range, and detects deviation. In a case where the deviation is detected, the deviation detection unit 45 acquires deviation information regarding the deviation such as how much the position of the star 62 indicating the latest state deviates from the normal range in the graph 60.

The thick line 61 in the graph 60 is, for instance, a boundary line that distinguishes between an area having the density equal to or more than a threshold and an area having the density less than the threshold, and can be arbitrarily set. That is, the boundary line on the life map used at the time of deviation detection can be arbitrarily set.

The risk prediction unit 46 predicts a risk of degradation of the quality of sleep of the subject, based on the tendency of the lifestyle, and the latest state of the subject. Specifically, the risk prediction unit 46 predicts the risk, based on the deviation information. For instance, the server 1 predicts that the risk is high in a case where the latest state of the subject deviates from the normal state. The server 1 may predict the risk in consideration of subjective evaluation of the quality of sleep, the magnitude of deviation, and the like. The risk may be represented in any stage or may be represented numerically.

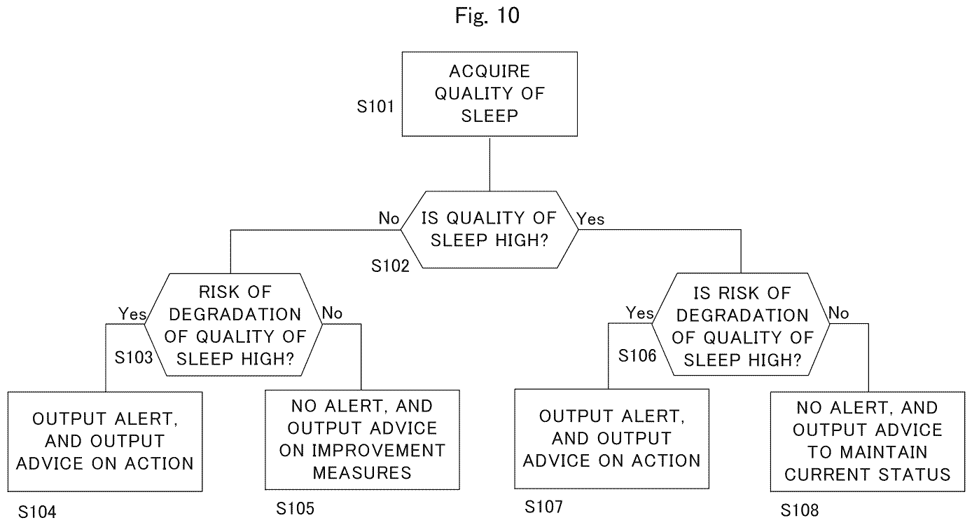

The feedback unit 47 provides feedback to the subject in a case where the risk of degradation of the quality of sleep of the subject is equal to or more than a threshold. FIG. 10 is a flowchart illustrating an example of processing by the feedback unit 47. As illustrated in FIG. 10, the feedback unit 47 acquires the subjective evaluation of the quality of sleep from the latest life log of the subject (step S101). Next, the feedback unit 47 determines whether the quality of sleep is high (step S102). A case where the quality of sleep is high is, for instance, a case where “1 very good” or “2 good” is input as the subjective evaluation. On the other hand, a case where the quality of sleep is low is, for instance, a case where “3 average”, “4 poor”, or “5 very poor” is input as the subjective evaluation.

In a case where it is determined that the quality of sleep is low (step S102; No), the feedback unit 47 determines whether the risk of degradation of the quality of sleep is high (step S103). In a case where the risk is equal to or more than the threshold and it is determined that the risk is high (step S103; Yes), the feedback unit 47 outputs an alert for notifying the subject that there is a high possibility that the quality of sleep will be low, and outputs advice for proposing a recommended action (step S104). On the other hand, in a case where the risk is less than the threshold and it is determined that the risk is low (step S103; No), the feedback unit 47 does not output the alert but outputs advice for improving the quality of sleep (step S105).

In a case where it is determined that the quality of sleep is high (step S102; Yes), the feedback unit 47 determines whether the risk of degradation of the quality of sleep is high (step S106). In a case where the risk is equal to or more than the threshold and it is determined that the risk is high (step S106; Yes), the feedback unit 47 outputs an alert for notifying the subject that there is a high possibility that the quality of sleep will be low, and outputs advice for proposing a recommended action (step S107). On the other hand, in a case where the risk is less than the threshold and it is determined that the risk is low (step S106; No), the feedback unit 47 does not output the alert but outputs advice for recommending maintaining the current state.

As illustrated in FIG. 5, the feedback unit 47 includes an alert output unit 51 and an action proposal unit 52. For instance, in a case where the risk is equal to or more than the threshold, the alert output unit 51 outputs, to the subject terminal 2, an alert for notifying the subject that there is a high possibility that the quality of sleep will be low. In a case where the risk is equal to or more than the threshold or the subjective evaluation of the quality of sleep is low, the action proposal unit 52 proposes a recommended action, based on a visualization map to be described later, and the latest state of the subject.

Based on the life log, the visualization map creation unit 48 creates a visualization map visualizing a relationship between the tendency of the lifestyle and the subjective evaluation of the quality of sleep. Specifically, the visualization map creation unit 48 creates a visualization map representing the density of points by concentration with any parameters as axes, based on a life log having an equivalent subjective evaluation of the quality of sleep. According to the visualization map, it is possible to visualize in which life log the subjective evaluation of the quality of sleep is good or in which life log the subjective evaluation of the quality of sleep is poor. With the visualization map, if a relationship between the own life log and the quality of sleep is known, in a case where the quality of sleep of the subject is likely to be low, the server 1 can take measures such as presenting a recommended action to the subject ahead of time.

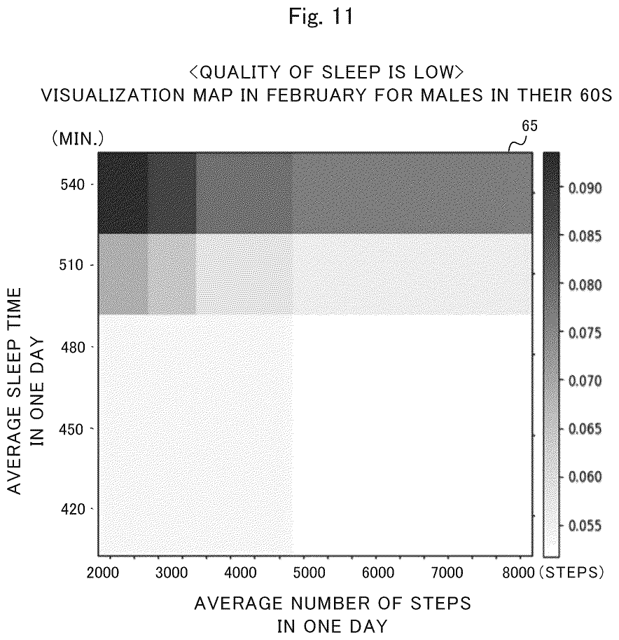

FIG. 11 illustrates an example of a visualization map with the average number of steps and the average sleep time as axes. A visualization map 65 illustrated in FIG. 11 is a graph visualizing a probability that the quality of sleep is low in February for males in their 60s who are of the same age and the same sex as the subject.

Although FIG. 11 illustrates the visualization map created based on life logs of users who have evaluated that the quality of sleep is low, the visualization map is not limited thereto, and may be created based on life logs of users who have evaluated that the quality of sleep is high. In this case, the visualization map is a graph visualizing a probability that the quality of sleep is high in February for males in their 60s who are of the same age and the same sex as the subject. The visualization map may be a graph visualizing a probability that the quality of sleep is low or a probability that the quality of sleep is high in February for the subject, created based on the life logs of the subject himself/herself in the past.

The action proposal unit 52 proposes a recommended action, based on the visualization map and the latest state of the subject. Specifically, as a recommended action of the subject, the action proposal unit 52 proposes in which order actions regarding which parameters as axes of the visualization map are to be performed, and transmits and displays proposal content to the subject terminal 2.

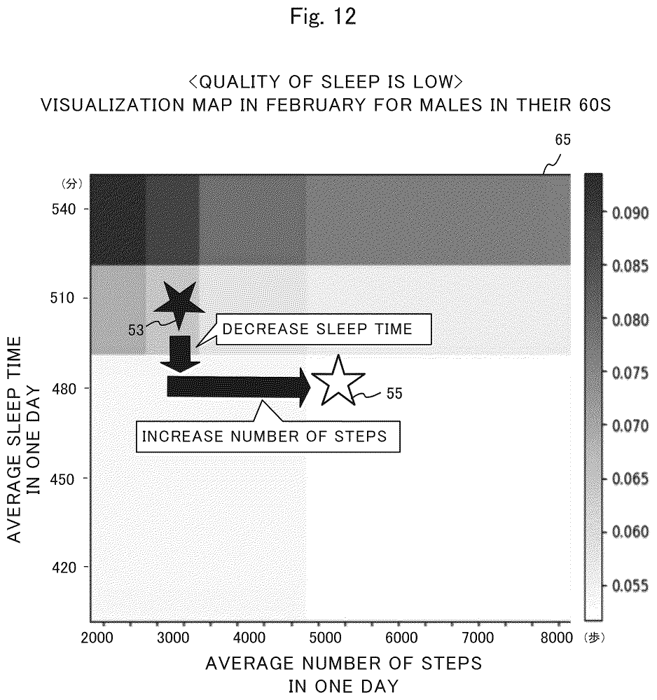

FIG. 12 is a diagram for explaining an action proposal based on the visualization map. For instance, in the visualization map 65 illustrated in FIG. 12, the action proposal unit 52 sets, as a target, a star 55 that is at a position having a least frequent subjective evaluation that the quality of sleep is low and proposes an action for reaching the star 55 as the target from a star 53 that is at a position indicating the latest state of the subject. As illustrated in FIG. 12, for reaching the star 55 from the star 53, it is necessary to increase the number of steps of the subject in one day and decrease the sleep time in one day so that the average number of steps and the average sleep time indicated by a position of the star 55 are obtained.

In a case where the visualization map is a graph visualizing a probability that the quality of sleep is high, the action proposal unit 52 sets, as a target, a position having a most frequent subjective evaluation that the quality of sleep is high, and proposes an action for reaching the position as the target from a position indicating the latest state of the subject.

The action recording unit 49 temporarily records, for instance, in the memory 13 or the like, an action actually executed by the subject, based on the life log of the subject within a preset period after the recommended action is proposed.

The execution determination unit 50 compares the action executed by the subject with the action proposed to the subject, and determines whether the subject has executed the proposed action.

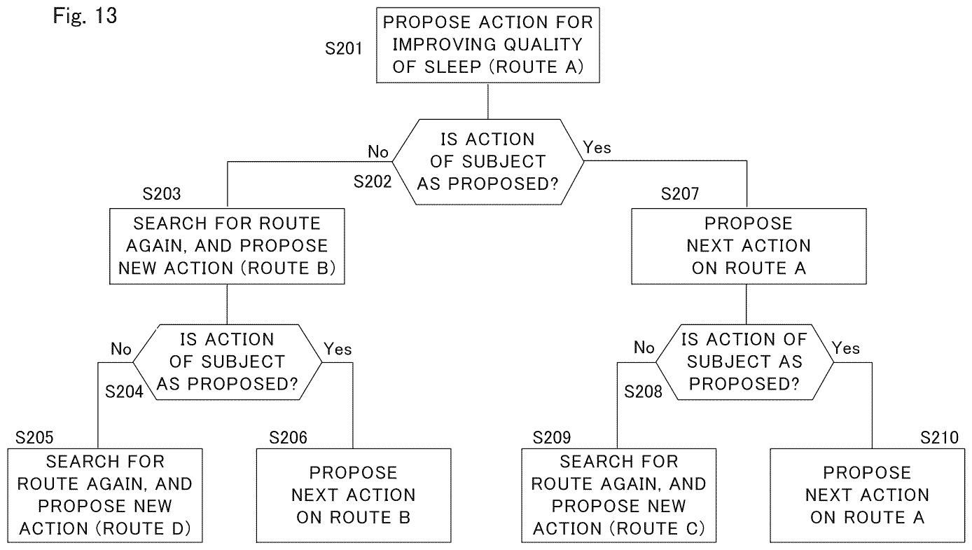

The action proposal unit 52 proposes a next action for improving the quality of sleep, based on a determination result as to whether the subject has executed the proposed action, the visualization map, and the latest state of the subject. FIG. 13 is a flowchart illustrating an example of processing by the action proposal unit 52. As illustrated in FIG. 13, the action proposal unit 52 first proposes a recommended action of the subject, based on the visualization map and the latest state of the subject (step S201).

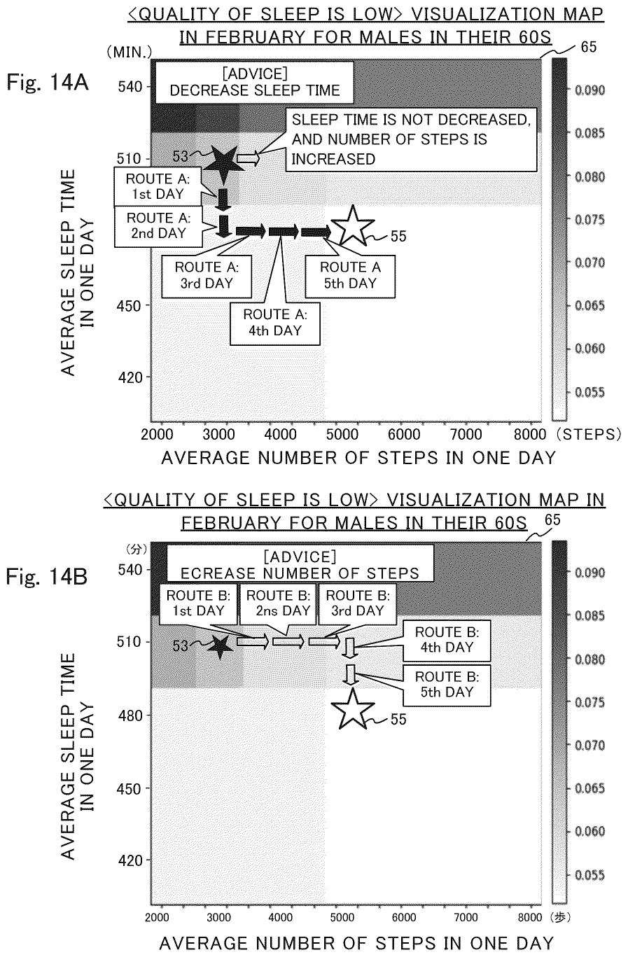

FIGS. 14A and 14B are diagrams for explaining a plurality of routes regarding an action for reaching a target. For instance, the action proposal unit 52 proposes, as an action for reaching the star 55 as the target from the star 53 indicating the latest state of the subject in the visualization map 65, a route A in which the sleep time is first decreased and then the number of steps is increased as indicated by a black arrow in FIG. 14A. One arrow corresponds to an action in one day, and in the example in FIG. 14A, the action proposal unit 52 proposes an action of decreasing the sleep time in one day for two days of the first day and the second day on the route A, and increasing the number of steps in one day for three days of the third day to the fifth day on the route A.

For instance, the action proposal unit 52 may transmit advice “decrease the sleep time” of the first day to the subject terminal 2 to display the advice, or may transmit information regarding the visualization map 65, the star 53, the star 55, and the arrow to the subject terminal 2 to display the information in addition to the advice. The advice to propose the action may be only the action on the next day, that is, the first day on the route A, or may be all actions from the first day to the fifth day on the route A.

If the action proposal unit 52 proposes the first action in step S201, the execution determination unit 50 determines whether the action of the subject is as proposed (step S202). Specifically, the execution determination unit 50 compares the actual action of the subject recorded by the action recording unit 49 with the first action proposed by the action proposal unit 52, and determines whether the subject has executed the proposed action.

For instance, in a case where the actual action of the subject recorded by the action recording unit 49 is “increase the number of steps” as indicated by a white arrow in FIG. 14A, since the first action proposed by the action proposal unit 52 is “decrease the sleep time”, the execution determination unit 50 determines that the action of the subject is not as proposed (step S202; No).

In a case where the action of the subject is not as proposed (step S202; No), the action proposal unit 52 searches for a route again and proposes a new action (step S203). As illustrated in FIG. 14B, the action proposal unit 52 searches for a route again, based on the actually executed action “increase the number of steps” of the subject, and proposes an action in a route B in which the number of steps is increased for the first three days and the sleep time is decreased for the subsequent two days, as an action for reaching the star 55 as the target.

The action proposal unit 52 may, for instance, transmit advice “increase the number of steps” to the subject terminal 2 to display the advice, or may transmit information regarding the visualization map 65, the star 53, the star 55, and the arrow to the subject terminal 2 to display the information in addition to the advice. The advice to propose the action may be only the action on the next day, that is, the second day on the route B, or may be all actions from the third day to the fifth day on the route B.

If the action proposal unit 52 proposes the next action in step S203, the execution determination unit 50 determines whether the action of the subject is as proposed (step S204). In a case where the action of the subject is not as proposed (step S204; No), the action proposal unit 52 searches for a route again and proposes a new action (step S205). Specifically, the action proposal unit 52 proposes a new action of a route D, based on the actual action of the subject. On the other hand, in a case where the action of the subject is as proposed (step S204; Yes), the action proposal unit 52 proposes the next action on the route B (step S206). As illustrated in FIG. 14B, in a case where the actual action of the subject is “increase the number of steps” as proposed, the action proposal unit 52 proposes an action “further increase the number of steps”, which is the action on the third day on the route B.

In the processing of step S202, in a case where the action of the subject is as proposed (step S202; Yes), the action proposal unit 52 proposes the next action on the route A (step S207). As illustrated in FIG. 14A, in a case where the actual action of the subject is “decrease the sleep time” as proposed, the action proposal unit 52 proposes an action “further decrease the sleep time”, which is the action on the second day on the route A.

If the action proposal unit 52 proposes the next action on the route A in step S207, the execution determination unit 50 determines whether the action of the subject is as proposed (step S208). In a case where the action of the subject is not as proposed (step S208; No), the action proposal unit 52 searches for a route again and proposes a new action (step S209). Specifically, the action proposal unit 52 proposes a new action in a route C, based on the actual action of the subject. On the other hand, in a case where the action of the subject is as proposed (step S208; Yes), the action proposal unit 52 proposes the next action on the route A (step S210). As illustrated in FIG. 14A, in a case where the actual action in the subject is “decrease the sleep time” as proposed, the action proposal unit 52 proposes an action “increase the number of steps”, which is an action on the third day on the route A.

As described above, the action proposal unit 52 proposes an action with a position where the quality of sleep is statistically high as a target, but can propose an appropriate action to the subject by updating an action to be proposed next as needed according to the action actually executed by the subject after the proposal. In other words, the action proposal unit 52 can change the recommended action according to the action actually executed by the subject after the proposal.

For reaching the position as the target from the position indicating the latest state of the subject, it is desirable to determine the order of actions, such as whether to decrease the sleep after increasing the number of steps, or whether to increase the sleep after decreasing the number of steps, according to the density of points indicating the users, based on the visualization map as illustrated in FIG. 14A. For instance, by proposing the order of actions so that the action is performed from a position where the density of points indicating the users is high to a position where the density is low, it is possible to first propose an action with many users and few hurdles. As a result, it is possible to reduce adverse effects such as difficulty in performing an action for improving sleep causes the action to be stopped.

In the above configuration, the life log acquisition unit 42, the life map creation unit 43, the state estimation unit 44, the deviation detection unit 45, the risk prediction unit 46, the feedback unit 47, the visualization map creation unit 48, the action recording unit 49, and the execution determination unit 50 of the server 1 are an example of a life log acquisition means, a life map creation means, a state estimation means, a deviation detection means, a risk prediction means, a feedback means, a visualization map creation means, an action recording means, and an execution determination means, respectively, of the present disclosure. The alert output unit 51 and the action proposal unit 52 included in the feedback unit 47 are an example of an alert output means and an action proposal means.

### Feedback Processing

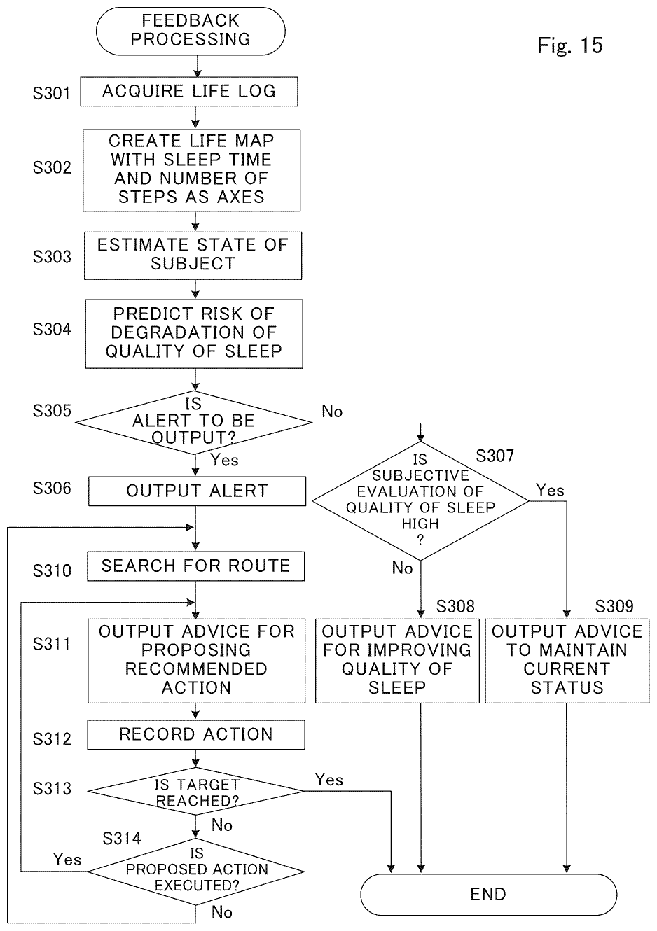

Next, feedback processing by the server 1 will be described. FIG. 15 is a flowchart illustrating an example of the feedback processing by the server 1. This processing is implemented by the processor 12 illustrated in FIG. 2A executing a program prepared in advance.

First, the server 1 acquires, from the life log DB 32, life logs of the subject and users of the same age and the same sex as the subject (step S301). Next, the server 1 creates a life map with, for instance, the average number of steps as the x axis and the average sleep time as the y axis, based on statistical information on the acquired life log (step S302). Next, the server 1 estimates the latest state of the subject based on the latest life log of the subject (step S303).

The server 1 predicts a risk of degradation of the quality of sleep of the subject, based on the life map and the latest state of the subject (step S304). Next, the server 1 determines whether to output an alert (step S305). In a case where the risk is equal to or more than the threshold, the server 1 determines to output the alert (step S305; Yes), and outputs, to the subject terminal 2, the alert notifying that the risk of degradation of the quality of sleep is high (step S306).

On the other hand, in a case where the risk is less than the threshold, the server 1 determines not to output the alert (step S305; No), and determines whether the latest subjective evaluation of the quality of sleep by the subject is good (step S307). In a case where the subjective evaluation of the quality of sleep is good (step S307; Yes), the server 1 outputs, to the subject terminal 2, advice indicating that it is desirable to maintain the current state (step S309), and ends the feedback processing. On the other hand, in a case where the subjective evaluation of the quality of sleep is poor (step S307; No), the server 1 outputs, to the subject terminal 2, advice for improving the quality of sleep, such as exposure to sunlight or moderate stretching at the time of getting up (step S308), and ends the feedback processing.

After outputting the alert by the processing of step S306, the server 1 creates a visualization map, and searches for a route for improving the quality of sleep on the visualization map, based on the latest state of the subject (step S310). Next, the server 1 outputs advice for proposing a recommended action to the subject terminal 2, based on the route for which the search has been performed (step S311). Next, the server 1 records the action executed by the subject, based on the life log of the subject within a preset period after proposing the recommended action (step S312).

Next, the server 1 determines whether a target has been reached, based on the action executed by the subject (step S313). Specifically, based on the latest life log of the subject, the server 1 determines whether the number of steps and the sleep time indicated by the latest state of the subject have reached the number of steps and the sleep time indicated by a position of the target on the visualization map.

In a case where it is determined that the target has not been reached (step S313; No), the server 1 compares the action executed by the subject with the action proposed to the subject, and determines whether the subject has executed the proposed action (step S314). In a case where the subject has executed the proposed action (step S314; Yes), the server 1 returns to the processing of step S311, and proposes a next recommended action, based on the route for which the search has been performed earlier (step S311).

On the other hand, in a case where the subject has not executed the proposed action (step S314; No), the server 1 returns to the processing of step S310, and searches for a route for improving the quality of sleep again on the visualization map, based on the latest state of the subject (step S310). Next, the server 1 proposes a next recommended action, based on the route for which the search has been performed again (step S311).

In the processing of step S313, in a case where it is determined that the target has been reached (step S313; Yes), the server 1 completes the feedback processing.

In the feedback processing illustrated in FIG. 15, in a case where the risk of degradation of the quality of sleep is less than the threshold, but the subjective evaluation of the quality of sleep is poor, advice such as exposure to sunlight at the time of getting up is output; however, the present disclosure is not limited to this, and the processing may proceed to step S310, and advice proposing a recommended action may be output based on the visualized map and the latest state of the subject.

In the feedback processing illustrated in FIG. 15, in the processing of steps S310 and S311, if the risk is equal to or more than the threshold regardless of the subjective evaluation of the quality of sleep by the subject, the recommended action is proposed and output to the subject terminal 2 as the advice; however, the present disclosure is not limited to this, and in a case where the subjective evaluation of the quality of sleep by the subject is good, the server 1 may output advice such as “The feeling of quality of sleep seems to be good, but the risk of degradation of the quality of sleep is high, so be careful.” instead of the recommended action.

According to such a risk prediction system 100, it is possible to predict the risk of degradation of the quality of sleep of the subject, based on the life log, and not only in a case where the subjective evaluation of the quality of sleep by the subject is low but also in a case where the subjective evaluation of the quality of sleep by the subject is high, it is possible to provide, to the subject, feedback such as an alert or advice if the risk is equal to or more than the threshold. That is, it is possible to notify the subject of the risk of degradation of the quality of sleep, and support health care of the subject.

In a case where the risk is equal to or more than the threshold or in a case where the subjective evaluation of the quality of sleep is low, the risk prediction system 100 can plan what kind of behavior change is to be performed for improving the quality of sleep, based on the life log, and present appropriate advice to the subject. Specifically, if it is detected that the state of the subject deviates from the normal state, based on the life map, and the latest life log of the subject, the risk prediction system 100 can present a specific action for recovering from the deviation as advice. The risk prediction system 100 can determine whether the subject has executed an action according to the advice, and appropriately update subsequent advice content according to a determination result. According to this, the risk prediction system 100 can present appropriate advice to the subject who cannot easily execute the action according to the advice at any time.

### First Modification Example

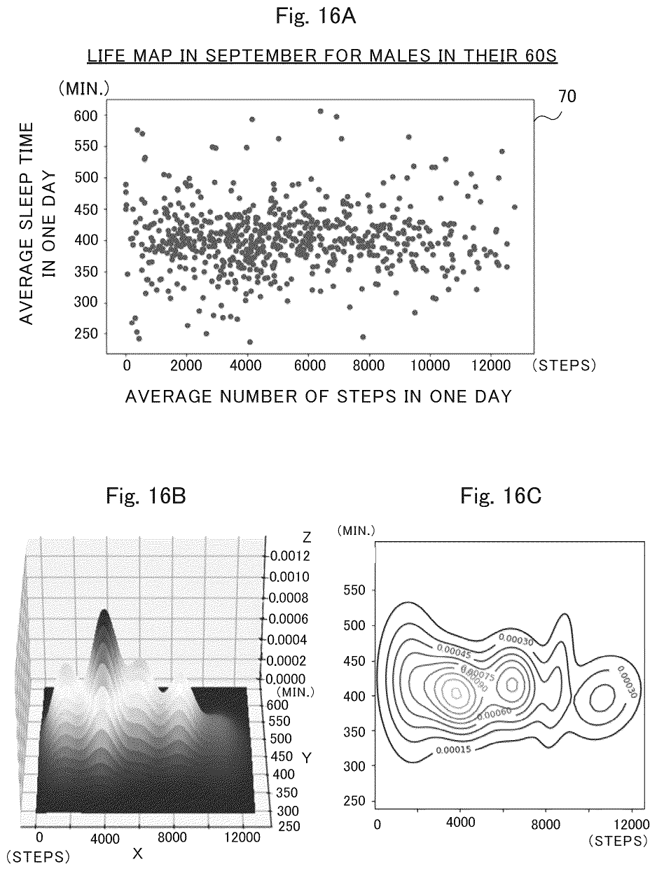

The life map illustrated in FIG. 6 is a life map created based on the life log in February, but the present disclosure is not limited thereto, and it is possible to set arbitrarily that the life map is created based on the life log of which period. FIG. 16A is a life map created based on life logs in September for males in their 60s of the same age and the same sex as the subject. Comparing a life map 70 in September illustrated in FIG. 16A with the life map 40 in February illustrated in FIG. 6, it can be seen that there are many users whose average number of steps is slightly smaller in the life map in September than in February. As described above, since the boundary line of deviation detection and the proposed recommended action also change with the life log that changes depending on the temperature and the climate, it is desirable to create the life map based on the life log at an appropriate period.

FIG. 16B is an example in which the life map 70 is converted into a three-dimensional graph with the density of points indicating the users as the Z axis. FIG. 16C is an example in which the life map 70 is converted into a graph representing the density of points indicating the users with contour lines.

### Second Modification Example

The life map and the visualization map are created with two or more parameters representing the tendency of the lifestyle as respective axes, based on the life log. It is considered that the temperature and the atmospheric pressure affect autonomic nerves and greatly affect the quality of sleep. For that reason, in addition to the life log, weather information regarding weather such as temperature and atmospheric pressure in one day may be stored in a predetermined DB, and may be used as parameters serving as axes of the life map or the visualization map. In this case, the life map creation unit 43 and the visualization map creation unit 48 create a life map and a visualization map using change in the weather, based on the life log and the weather information, and the action proposal unit 52 proposes a recommended action reflecting the change in the weather. As a result, the risk prediction system 100 can predict the risk in consideration of the weather such as a typhoon in which the atmospheric pressure changes, and provide appropriate feedback to the subject according to a prediction result.

The life log and the weather information may not be stored in different DBs, and the weather information may be handled as a part of the life log.

### Third Modification Example

The risk of degradation of the quality of sleep may be predicted by use of a risk prediction model that is a machine learning model. For instance, the risk prediction unit 46 may construct a risk prediction model for outputting an optimized numerical value indicating a risk if the life map and data regarding the latest state of the subject are input. Teacher data is used to construct (generate) the risk prediction model. The teacher data is data in which input data input in training of the risk prediction model is associated with ground truth data corresponding to the input data. The input data is data regarding various life maps and the latest state of the subject, and the ground truth data is a numerical value indicating a risk. The risk prediction unit 46 causes the risk prediction model to perform learning for outputting a numerical value indicating a risk, based on the life map and the data regarding the latest state of the subject input as input data. Examples of the machine learning method include a model using a neural network. According to this, the risk prediction unit 46 can set the numerical value indicating the risk output by the risk prediction model as the risk by the subject.

### Fourth Modification Example

In the above example embodiment, the subject uses the subject terminal 2, but the present disclosure is not limited to this, and the subject may use a subject terminal having a function of the server 1. In this case, the subject terminal can predict a risk by executing the feedback processing performed by the server 1, and provide appropriate feedback to the subject according to a prediction result.

### Second Example Embodiment

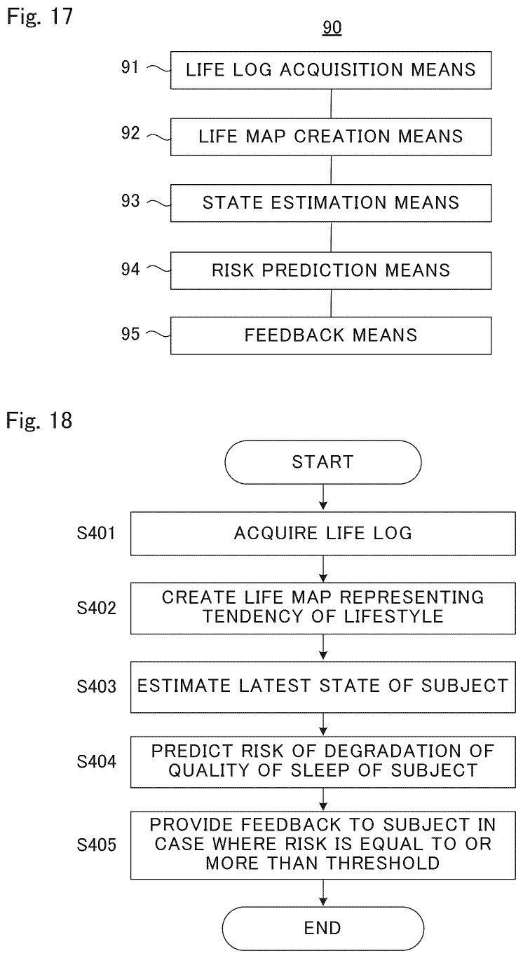

FIG. 17 is a block diagram illustrating an example of a functional configuration of the risk prediction device in the present disclosure. A risk prediction device 90 includes a life log acquisition means 91, a life map creation means 92, a state estimation means 93, a risk prediction means 94, and a feedback means 95.

FIG. 18 is a flowchart illustrating an example of processing by the risk prediction device 90. The life log acquisition means 91 acquires a life log including the quality of sleep (step S401). The life map creation means 92 creates a life map representing the tendency of the lifestyle, based on the life log (step S402). The state estimation means 93 estimates the latest state of the subject, based on the latest life log of the subject (step S403). The risk prediction means 94 predicts a risk of degradation of the quality of sleep of the subject, based on the life map and the latest state of the subject. In a case where the risk is equal to or more than the threshold, the feedback means 95 provides feedback to the subject (step S405).

According to the first example embodiment (including the first modification example to the fourth modification example) and the second example embodiment, it is possible to supports users in making decisions concerning their sleep.

Some or all of the above example embodiments (including the modification example, the same applies hereinafter) may also be described as the following supplementary notes, but are not limited to the following supplementary notes.

### Supplementary Note 1

A risk prediction device including
a life log acquisition means configured to acquire a life log including quality of sleep,
a life map creation means configured to create a life map representing a tendency of a lifestyle, based on the life log,
a state estimation means configured to estimate a latest state of a subject, based on a latest life log of the subject,
a risk prediction means configured to predict a risk of degradation of quality of sleep of the subject, based on the life map and the latest state of the subject, and
a feedback means configured to provide feedback to the subject in a case where the risk is equal to or more than a threshold.

### Supplementary Note 2

The risk prediction device according to Supplementary Note 1, including
a deviation detection means configured to detect that the latest state of the subject deviates from a normal state of the subject in the life map and acquiring deviation information regarding deviation, in which
the life log acquisition means acquires life logs of the subject at a plurality of time points in the past,
the life map creation means creates a life map representing a tendency of a lifestyle of the subject, based on the life logs of the subject in the past, and
the risk prediction means predicts the risk, based on the deviation information.

### Supplementary Note 3

The risk prediction device according to Supplementary Note 1, in which
the subject is one of a plurality of users,
a deviation detection means is included for detecting that the latest state of the subject deviates from a normal state of users of a sex and an age identical to those of the subject in the life map and acquiring deviation information regarding deviation,
the life log acquisition means acquires life logs of the plurality of users at a plurality of time points in the past,
the life map creation means creates a life map representing a tendency of a lifestyle of the users of the sex and the age identical to those of the subject, based on the life logs of the plurality of users, and
the risk prediction means predicts the risk, based on the deviation information.

### Supplementary Note 4

The risk prediction device according to Supplementary Note 1, in which the feedback means outputs an alert to a terminal used by the subject in a case where the risk is equal to or more than the threshold.

### Supplementary Note 5

The risk prediction device according to Supplementary Note 4, including
a visualization map creation means configured to create a visualization map visualizing a relationship between the tendency of the lifestyle and subjective evaluation of the quality of sleep, based on the life log, and
an action proposal means configured to propose an action for improving the quality of sleep, based on the visualization map and the latest state of the subject in a case where the risk is equal to or more than the threshold,
in which the feedback means outputs advice indicating the action for improving the quality of sleep to the terminal used by the subject.

### Supplementary Note 6

The risk prediction device according to Supplementary Note 5, including
an action recording means configured to record an action executed by the subject, based on a life log of the subject within a preset period after the action for improving the quality of sleep is proposed, and
an execution determination means configured to compare an action executed by the subject with an action proposed to the subject and performs determination of whether the subject has executed the action proposed,
in which the action proposal means proposes a next action for improving the quality of sleep, based on a result of the determination, the visualization map, and the latest state of the subject.

### Supplementary Note 7

The risk prediction device according to Supplementary Note 6, in which
the life log includes information regarding a plurality of parameters representing the tendency of the lifestyle,
the life map and the visualization map each have two or more parameters as axes,
the parameters include information regarding weather in one day,
the life map creation means creates a life map using a change in the weather, and
the action proposal means proposes an action reflecting the change in the weather.

### Supplementary Note 8

The risk prediction device according to Supplementary Note 1, in which the risk prediction means predicts the risk of degradation of the quality of sleep of the subject by using a machine learning model trained to output an optimized numerical value indicating the risk in response to input of the life map and data regarding the latest state of the subject.

### Supplementary Note 9

A risk prediction method executed by a risk prediction device, the risk prediction method including
acquiring a life log including quality of sleep,
creating a life map representing a tendency of a lifestyle, based on the life log,
estimating a latest state of a subject, based on a latest life log of the subject,
predicting a risk of degradation of quality of sleep of the subject, based on the life map and the latest state of the subject, and
providing feedback to the subject in a case where the risk is equal to or more than a threshold.

### Supplementary Note 10

A program executed by a risk prediction device including a computer, the program causing the computer to execute processing of
acquiring a life log including quality of sleep,
creating a life map representing a tendency of a lifestyle, based on the life log,
estimating a latest state of a subject, based on a latest life log of the subject,
predicting a risk of degradation of quality of sleep of the subject, based on the life map and the latest state of the subject, and
providing feedback to the subject in a case where the risk is equal to or more than a threshold.

### Supplementary Note 11

The risk prediction device according to Supplementary Note 1, in which the life map creation means creates the life map, based on a life log in a case where a feeling that the quality of sleep is high is obtained.

### Supplementary Note 12

The risk prediction device according to Supplementary Note 6, in which
the life log includes information regarding a plurality of parameters representing the tendency of the lifestyle,
the life map and the visualization map each have two or more parameters as axes, and
the action proposal means proposes in which order actions regarding which parameters are to be performed.

### Supplementary Note 13

The risk prediction device according to Supplementary Note 12, in which the parameters are sleep time and the number of steps.

### Supplementary Note 1

The risk prediction device according to Supplementary Note 12, in which the action proposal means sets, as a target, a position having a most frequent subjective evaluation that the quality of sleep is high in the visualization map, and proposes in which order actions regarding which parameters are to be performed for reaching the position as the target from a position indicating the latest state of the subject.

### Supplementary Note 15

The risk prediction device according to Supplementary Note 12, in which the action proposal means sets, as a target, a position having a least frequent subjective evaluation that the quality of sleep is low in the visualization map, and proposes in which order actions regarding which parameters are to be performed for reaching the position as the target from a position indicating the latest state of the subject.

Some or all of the configurations described in Supplementary Notes 2 to 8 and Supplementary Notes 11 to 15 dependent on the above-described Supplementary Note 1 can also be dependent on Supplementary Notes 9 and 10 by a dependency relationship similar to that in Supplementary Notes 2 to 8 and Supplementary Notes 11 to 15. Some or all of the configurations described as the Supplementary Notes can be similarly dependent on not only the Supplementary Notes 1, 9, and 10, but also various pieces of hardware and software, and various recording means or systems for recording software without departing from the above-described example embodiments.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. That is, it is a matter of course that the present disclosure includes various modifications and corrections that can be made by those of ordinary skill in the art in accordance with the entire disclosure including the claims and the technical idea.

### DESCRIPTION OF SYMBOLS

1 Server
2 Terminal
11, 21 Interface
12, 22 Processor
13, 23 Memory
14, 24 Recording medium
15, 25 Display unit
16, 26 Input unit
41 Personal information acquisition unit
42 Life log acquisition unit
43 Life map creation unit
44 State estimation unit
45 Deviation detection unit
46 Risk prediction unit
47 Feedback unit
48 Visualization map creation unit
49 Action recording unit
50. Execution determination unit

1. A risk prediction device comprising:
at least one interface configured to communicate with a subject terminal used by a subject;
at least one memory configured to store instructions; and
at least one processor configured to execute the instructions to:
acquire, via the at least one interface, a life log for the subject from the subject terminal, the life log including at least quality-of-sleep information relating to sleep of the subject and lifestyle information for a plurality of days;
create, based on the life log, a life map representing a tendency of a lifestyle of the subject in a coordinate space having, as axes, a plurality of lifestyle-related parameters calculated from the lifestyle information;
estimate a latest state of the subject by calculating, from a latest life log of the subject for a predetermined period, values of the plurality of lifestyle-related parameters and mapping the latest state to a position in the coordinate space of the life map;
predict a risk of degradation of quality of sleep of the subject, based on a positional relationship between the position of the latest state and a region in the life map that is associated with a predetermined condition on the quality-of-sleep information included in the life log; and
control the subject terminal, via the at least one interface, to provide feedback to the subject in a case where the risk is equal to or more than a threshold, by causing the subject terminal to display at least one of an alert indicating a high possibility of low quality of sleep will be and a recommended action for changing at least one of the plurality of lifestyle-related parameters so that the risk is reduced.

2. The risk prediction device according to claim 1, wherein the at least one processor is further configured to:
detect that a position of the latest state mapped to the coordinate space of the life map deviates from a normal range for the subject in the life map and to acquire deviation information regarding the deviation, wherein
the at least one processor acquires life logs of the subject at a plurality of time points in the past different from the predetermined period,
the at least one processor creates, based on the life logs of the subject in the past, the life map representing the tendency of the lifestyle of the subject in the coordinate space, and
the at least one processor predicts the risk based on the deviation information.

3. The risk prediction device according to claim 1, wherein
the subject is one of a plurality of users,
the at least one processor is further configured to:
detect that a position of the latest state mapped to the coordinate space of the life map deviates from a normal range for users having a sex and an age identical to those of the subject and to acquire deviation information regarding the deviation, wherein
the at least one processor acquires life logs of the plurality of users at a plurality of time points in the past,
the at least one processor creates, based on the life logs of the plurality of users, another life map representing a tendency of a lifestyle of the users having the sex and the age identical to those of the subject in the coordinate space, and
the at least one processor predicts the risk based on the deviation information.

4. The risk prediction device according to claim 1, wherein
the at least one processor controls the subject terminal, via the at least one interface, to display, as part of the feedback, the alert in a case where the risk is equal to or more than the threshold.

5. The risk prediction device according to claim 4, wherein the at least one processor is further configured to:
create, based on the life log, a visualization map visualizing a relationship between the tendency of the lifestyle represented by the life map and subjective evaluation of the quality of sleep included in the life log; and
propose a recommended action for improving the quality of sleep based on the visualization map and a position of the latest state in the coordinate space in a case where the risk is equal to or more than the threshold,
wherein the at least one processor controls the subject terminal, via the at least one interface, to display advice indicating the recommended action as part of the feedback.

6. The risk prediction device according to claim 5, wherein the at least one processor is further configured to:
record, based on a life log of the subject within a preset period after the recommended action for improving the quality of sleep is proposed, an action executed by the subject; and
compare the action executed by the subject with the recommended action proposed to the subject and perform determination of whether the subject has executed the recommended action, and
propose a next recommended action for improving the quality of sleep based on a result of the determination, the visualization map, and an updated position of the latest state in the coordinate space.

7. The risk prediction device according to claim 6, wherein
the life log includes information regarding a plurality of parameters representing the tendency of the lifestyle,
the life map and the visualization map each have two or more parameters as axes,
the parameters include information regarding weather in one day,
the at least one processor creates the life map such that the coordinate space includes, as one of the axes, a parameter indicating a change in the weather, and
the at least one processor proposes the recommended action reflecting the change in the weather.

8. The risk prediction device according to claim 1, wherein
the at least one processor predicts the risk of degradation of the quality of sleep of the subject by using a machine learning model trained to output, as an optimized numerical value, the risk in response to input of data indicating at least a position of the latest state in the coordinate space of the life map and a region in the life map that is associated with the predetermined condition on the quality-of-sleep information.

9. The risk prediction device according to claim 1, wherein the recommended action is intended to support decision making of the subject in reaching a target regarding the quality of sleep.

10. A risk prediction method executed by a risk prediction device including at least one interface configured to communicate with a subject terminal used by a subject, at least one memory configured to store instructions, and at least one processor configured to execute the instructions, the risk prediction method comprising:
acquiring, via the at least one interface, a life log for the subject from the subject terminal, the life log including at least quality-of-sleep information relating to sleep of the subject and lifestyle information for a plurality of days;
creating, based on the life log, a life map representing a tendency of a lifestyle of the subject in a coordinate space having, as axes, a plurality of lifestyle-related parameters calculated from the lifestyle information;
estimating a latest state of the subject by calculating, from a latest life log of the subject for a predetermined period, values of the plurality of lifestyle-related parameters and mapping the latest state to a position in the coordinate space of the life map;
predicting a risk of degradation of quality of sleep of the subject, based on a positional relationship between the position of the latest state and a region in the life map that is associated with a predetermined condition on the quality-of-sleep information included in the life log; and
providing feedback to the subject in a case where the risk is equal to or more than a threshold, by controlling the subject terminal via the at least one interface to display at least one of an alert indicating a high probability that the quality of sleep will be low and a recommended action for changing at least one of the plurality of lifestyle-related parameters so that the risk is reduced.

11. The risk prediction method according to claim 10, further comprising:
acquiring life logs of the subject at a plurality of time points in the past different from the predetermined period;
creating, based on the life logs of the subject in the past, the life map representing the tendency of the lifestyle of the subject in the coordinate space; and
detecting that the position of the latest state mapped to the coordinate space deviates from a normal range for the subject in the life map and acquiring deviation information regarding the deviation,
wherein predicting the risk comprises predicting the risk based on the deviation information.

12. The risk prediction method according to claim 10, wherein the subject is one of a plurality of users, the risk prediction method further comprising:
acquiring life logs of the plurality of users at a plurality of time points in the past;
creating, based on the life logs of the plurality of users, another life map representing a tendency of a lifestyle of users having a sex and an age identical to those of the subject in the coordinate space; and
detecting that the position of the latest state mapped to the coordinate space deviates from a normal range for the users having the sex and the age identical to those of the subject and acquiring deviation information regarding the deviation,
wherein predicting the risk comprises predicting the risk based on the deviation information.

13. The risk prediction method according to claim 10, wherein
providing the feedback comprises causing the subject terminal, via the at least one interface, to display the alert, as part of the feedback, in a case where the risk is equal to or more than the threshold.

14. The risk prediction method according to claim 13, further comprising:
creating, based on the life log, a visualization map visualizing a relationship between the tendency of the lifestyle represented by the life map and subjective evaluation of the quality of sleep included in the life log; and
proposing the recommended action for improving the quality of sleep, based on the visualization map and the position of the latest state in the coordinate space in a case where the risk is equal to or more than the threshold,
wherein providing the feedback comprises causing the subject terminal, via the at least one interface, to display advice indicating the recommended action as part of the feedback.

15. The risk prediction method according to claim 14, further comprising:
recording, based on a life log of the subject within a preset period after the recommended action for improving the quality of sleep is proposed, an action executed by the subject; and
comparing the action executed by the subject with the recommended action proposed to the subject and performing determination of whether the subject has executed the recommended action,
wherein the risk prediction method further comprises proposing a next recommended action for improving the quality of sleep based on a result of the determination, the visualization map, and an updated position of the latest state in the coordinate space.

16. The risk prediction method according to claim 15, wherein
the life log includes information regarding a plurality of parameters representing the tendency of the lifestyle,
the life map and the visualization map each have two or more parameters as axes,
the parameters include information regarding weather in one day,
the creating of the life map comprises creating the life map such that the coordinate space includes, as one of the axes, a parameter indicating a change in the weather, and
the proposing of the recommended action comprises proposing the recommended action reflecting the change in the weather.

17. The risk prediction method according to claim 10, wherein
predicting the risk of degradation of the quality of sleep of the subject comprises using a machine learning model trained to output, as an optimized numerical value, the risk in response to input of data indicating at least the position of the latest state in the coordinate space of the life map and a region in the life map that is associated with the predetermined condition on the quality-of-sleep information.

18. The risk prediction method according to claim 10, wherein the recommended action is intended to support decision making of the subject in reaching a target regarding the quality of sleep.

19. A non-transitory computer-readable recording medium storing a program causing a computer of a risk prediction device including at least one interface configured to communicate with a subject terminal used by a subject, at least one memory configured to store instructions, and at least one processor configured to execute the instructions to execute processing of:
acquiring, via the at least one interface, a life log for the subject from the subject terminal, the life log including at least quality-of-sleep information relating to sleep of the subject and lifestyle information for a plurality of days;
creating, based on the life log, a life map representing a tendency of a lifestyle of the subject in a coordinate space having, as axes, a plurality of lifestyle-related parameters calculated from the lifestyle information;
estimating a latest state of the subject by calculating, from a latest life log of the subject for a predetermined period, values of the plurality of lifestyle-related parameters and mapping the latest state to a position in the coordinate space of the life map;
predicting a risk of degradation of quality of sleep of the subject, based on a positional relationship between the position of the latest state and a region in the life map that is associated with a predetermined condition on the quality-of-sleep information included in the life log; and
providing feedback to the subject in a case where the risk is equal to or more than a threshold, by controlling the subject terminal via the at least one interface to display at least one of an alert indicating a high probability that the quality of sleep will be low and a recommended action for changing at least one of the plurality of lifestyle-related parameters so that the risk is reduced.

20. The non-transitory computer-readable recording medium according to claim 19, wherein the recommended action is intended to support decision making of the subject in reaching a target regarding the quality of sleep.
